(19) United States
(12) Patent Application Publication
(10) Pub. No.: US 2006/0110426 A1
(43) Pub. Date: May 25, 2006

(54) Cohesive coprecipitates of sulfated polysaccharide and fibrillar protein and use thereof

(75) Inventors: Zvi Nevo, Herzliya (IL); Liliana Astachov, Ganei Yochanan (IL); Semion Rochkind, Ramat Aviv Gimel (IL); Abraham Shahar, Rehovot (IL)

Correspondence Address: HESLIN ROTHENBERG FARLEY & MESITI PC, 5 COLUMBIA CIRCLE, ALBANY, NY, 12203, US

(73) Assignee: NVR LABS, LTD., Ness Ziona (IL)

(21) Appl. No.: 10/529,830

(22) PCT Filed: Sep. 30, 2003
(86) PCT No.: PCT/IL03/00787

§ 371 Date: Oct. 7, 2005

(30) Foreign Application Priority Data
Sep. 30, 2002 (IL) 152030

(51) Int. Cl. A61K 35/12; A61F 2/00

(52) U.S. Cl. 424/423; 424/93.7

(57) ABSTRACT

The present invention concerns cohesive biopolymer gels comprising coprecipitates of sulfated polysaccharides and fibrillar proteins, exemplified by coprecipitates of dextran sulfate and gelatin, useful for clinical applications including as implants for tissue engineering as well as in biotechnology. The cohesive biopolymer gels according to the present invention may be used clinically either per se or as a scaffold for a cell-bearing implant, as a depot for sustained release of bioactive agents, or for research and biotechnology.

### FIELD OF THE INVENTION

The present invention relates to compositions comprising coprecipitates of sulfated polysaccharide and fibrillar protein, exemplified by a coprecipitate of dextran sulfate and gelatin, that form a cohesive biopolymer having unique physicochemical attributes useful as universal biomatrices or scaffolds for clinical applications including as implants for tissue engineering as well as in biotechnology. The matrices according to the present invention may be used clinically either per se or as a scaffold for a cell-bearing implant.

### BACKGROUND OF THE INVENTION

Matrices useful for guided tissue regeneration and/or as biocompatible surfaces useful for tissue culture or tissue implants are well known in the art. These matrices may therefore be considered as substrates for cell growth either in vitro or in vivo. Suitable matrices for tissue growth and/or regeneration and/or implants include both biodegradable and biostable entities. Among the many candidates that may serve as useful matrices claimed to support tissue growth or regeneration, are included gels, foams, sheets, and numerous porous particulate structures fabricated at different densities and in different forms and shapes.

In many instances the matrix may advantageously be composed of biopolymers, including polypeptides or proteins, as well as various polysaccharides, including proteoglycans, sulfated polyglycans and the like. In addition, these biopolymers may be either selected or manipulated in ways that affect their physicochemical properties. For example, biopolymers may be cross-linked either enzymatically, chemically or by other means, thereby providing greater or lesser degrees of rigidity or susceptibility to degradation.

Among the manifold natural polymers which have been disclosed to be useful for tissue engineering or culture, one can enumerate various constituents of the extracellular matrix including fibronectin, various types of collagen, and laminin, as well as keratin, fibrin and fibrinogen, hyaluronic acid, heparan sulfate, chondroitin sulfate and others.

U.S. Pat. Nos. 5,955,438 and 4,971,954 disclose collagen-based matrices cross-linked by sugars, useful for tissue regeneration.

U.S. Pat. No. 5,948,429 disclosing methods of making and using biopolymer foams comprising extracellular matrix particulates.

U.S. Pat. Nos. 6,083,383 and 5,411,885 disclose fibrin or fibrinogen glue and methods for using same. U.S. Pat. Nos. 5,279,825 and 5,173,295 disclose a method of enhancing the regeneration of injured nerves and adhesive pharmaceutical formulations comprising fibrin. U.S. Pat. No. 4,642,120 discloses the use of fibrin or fibrinogen glue in promoting repair of defects of cartilage and bone.

U.S. Pat. Nos. 6,124,265 and 6,110,487 disclose methods of making and cross-linking keratin-based films and sheets and of making porous keratin scaffolds and products of same.

Hyaluronic acid (HA) is a naturally occurring high molecular weight linear polymer belonging to the glycosaminoglycan family, composed of repeating units of glucuronic acid and N-acetyl glucosamine. HA readily forms hydrated gels which serve in vivo as space filling substance. The utility of hyaluronic acid as a beneficial component for supporting tissue growth is well established in the art, as exemplified in U.S. Pat. No. 5,942,499, which discloses methods of promoting bone growth with hyaluronic acid and growth factors. U.S. Pat. Nos. 5,128,326 and 5,783,691 disclose methods of producing and using cross-linked hyaluronans in promoting tissue repair and as reservoirs for bioactive agents including drugs or growth factors.

Laminin (LN) is an adhesive glycoprotein of high molecular weight, which is known as a major cell matrix binding component. U.S. Pat. Nos. 4,829,000 and 5,158,874 exemplify uses of gels or matrices comprising laminin.

WO 92/21354 discloses biocompatible anionic polymers that inhibit fibrosis, scar formation and surgical adhesions. Anionic polymers for use in the invention include but are not limited to natural proteoglycans, and the glycosaminoglycan (GAG) moieties of proteoglycans. The anionic polymers dextran sulfate and pentosan polysulfate are preferred, and according to a more preferred embodiment Dextran Sulfate, preferably with a molecular weight of 40,000 to 500,000 Daltons in which the sulfur content is greater than about 10% by weight is used.

U.S. Pat. No. 5,861,382 and U.S. Pat. No. 6,020,323 disclose substances comprising carboxylated or sulfated oligo-saccharides in substantially pure form, and methods of using same for the regulation of cytokine activity in a host.

One of the present inventors has previously disclosed (WO 01/02030) a device with a constant perfusion system for maintenance of viable cells, tissues and composite implants. That disclosure further concerns a scaffold which is used as a growth supportive base for various cells and tissue explants comprising naturally derived connective tissue or skeletal tissue, cross-linked with one of the following: HA, proteoglycans, GAGs, chondroitin sulfates, heparan sulfates, heparins and dextran sulfates.

Cross-linking between macromolecules of the extra-cellular matrix may occur naturally (Laurent, 1964; Wang and Bozos, 1985). In vitro, heparin was reported to interact with natural occurring mammalian's proteins such as amyloid (Cohlbery et. al., 2002) or S-100 (Robinson et. al., 2002).

Certain specific combinations of polysaccharides and fibrillar proteins have been used to promote cell growth. For example, the non-sulfated polysaccharide chitosan has been combined with gelatin as a scaffold supporting chondrocyte growth and differentiation in vitro (Risbud M. et al. 2001); vascular cells responded in vitro and in vivo to chitosan and dextran sulfate (Chupa et.al., 2000); and auricular chondrocytes of elastic cartilage were shown to grow in hydrogels of collagen and alginate, a non-sulfated polysaccharide composed of polymannuronic acid (de-Chalain et. al., 1999).

U.S. Pat. No. 4,280,954 discloses composite materials which are formed by contacting collagen with a mucopolysaccharide and subsequently covalently cross-linking the resultant polymer. U.S. Pat. No. 4,448,718 discloses that the cross-linking is performed by gaseous aldehyde, and U.S. Pat. No. 4,350,629 further discloses that if collagen fibrils are contacted with a cross-linking agent before being contacted with the glycosaminoglycan, the materials produced have extremely low levels of thrombogenicity

U.S. Pat. Nos. 5,866,165 and 5,972,385 disclose a method for preparing a matrix, the method comprising reacting a polysaccharide with an oxidizing agent to open sugar rings on the polysaccharide to form aldehyde groups, and reacting the resulted aldehyde groups to form covalent linkages to collagen, and the use of the matrix to support the growth of tissue, such as bone, cartilage or soft tissue. U.S. Pat. No. 6,309,670 discloses the use of this matrix, which further comprises a differentiation factor for the treatment of a bone tumor.

U.S. Pat. No. 6,624,245 discloses a composition prepared by admixture of individually reactive polymer components, wherein the admixture initiates rapid cross-linking and gel formation, wherein such compositions are suited for use in applications in which rapid adhesion to the tissue and gel formation is desired, including using the compositions as bioadhesives, for tissue augmentation, in the prevention of surgical adhesions, for coating surfaces of synthetic implants and the like.

Dextran sulfate alone was found to act as an antimicrobial agent (Christensen et al., 2001) and as prophylaxis for peritoneal cancer metastasis (Hagiwara et al., 2000). It was also used as an antifoam agent of proteins (Ibanoglu et. al., 2001)

Nowhere in the background art is it taught or suggested that biopolymers comprising fibrillar proteins coprecipitated with sulfated polysaccharides in general and dextran sulfate in particular, have novel physicochemical properties. Furthermore, the use of these scaffold matrices as an implant suitable for transplantation, per se or as cell bearing implants, has never been disclosed.

### SUMMARY OF THE INVENTION

It is an object of the present invention to provide a cohesive biopolymer gel that is biocompatible and affords a convenient environment for tissue repair. It is a further object of the present invention to provide a universal biopolymer scaffold suitable for many cell bearing implants which may conveniently be used either in vitro or in vivo. It is a further object of the present invention to provide a scaffold or gel which is useful for clinical applications due to its unique attributes of fostering tissue regeneration.

The cohesive biopolymer may be fabricated in the form of a gel, sleeve, cuff, sponge, membrane or any other shape useful as a scaffold for tissue repair.

These and other objects of the present invention are met by cohesive biopolymer gels comprising a coprecipitate of at least one sulfated polysaccharide and at least one fibrillar protein, wherein the coprecipitate is formed in the absence of an exogenous cross-linking agent in the presence of a volatile organic solvent. Surprisingly, the coprecipitation of a fibrillar protein with a polyanionic polysaccharide provides a gel with highly advantageous properties for use in vivo. According to some embodiments the polyanionic sulfated polysaccharide is selected from the group consisting of dextran sulfate, chondroitin sulfate, heparan sulfate, heparin, keratan sulfate, dermatan sulfate, as well as algal polyglycan sulfates, or synthetic sulfated polysaccharides, as are known in the art. According to some embodiments the fibrillar protein is selected from the group consisting of collagen, elastin, fibrin, albumin and gelatin.

According to one currently more preferred embodiment exemplified herein, dextran sulfate is coprecipitated with gelatin.

Though it is possible to use gelatin obtained from human collagen, more preferred are materials of non-human origin, due to safety concerns related to the use of collagens obtained from human sources. According to certain embodiments porcine or bovine gelatin are used to form the coprecipitates of the invention, though other mammalian species may also be used.

Any type of dextran sulfate may be employed according to the principles of the present invention, providing different biopolymer properties according to the molecular weight of the dextran sulfate used. According to one embodiment of the present invention the dextran sulfate has a molecular weight of from about 4,000 Dalton to about 500,000 Dalton.

According to some embodiments, dextran sulfate having high molecular weights are preferred, particularly dextran sulfate having a molecular weight of more than 300,000 Daltons, preferably more than 400,000 Dalton, most preferably dextran sulfate of about 500,000 Dalton.

According to alternative embodiments, dextran sulfate having low molecular weights are preferred, particularly dextran sulfate having a molecular weight below 10,000 Daltons, preferably below 8,000 Dalton, most preferably dextran sulfate of about 5,000 Dalton.

The properties of the cohesive biopolymer gel of the present invention are dependent on the conditions under which the dextran sulfate and the gelatin coprecipitate, particularly on the pH during the coprecipitation. According to one embodiment the coprecipitate is formed at a pH of at least 2 pH units above or below neutral pH.

According to some embodiments, high molecular weight dextran sulfate (i.e. of more than 300,000 Dalton) is coprecipitated with gelatin under acidic pH conditions. According to one preferred embodiment, the acidic pH conditions comprise a pH in the range of from about 2.0 to about 5.0, more preferably from about 2.0 to about 4.0.

According to alternative embodiments, low molecular weight dextran sulfate (i.e. dextran sulfate of bellow 10,000 Dalton) is coprecipitated with gelatin under basic pH conditions. According to one preferred embodiment, the basic pH conditions comprise a pH in the range of from about 8.0 to about 12.0, preferably from about 9.0 to about 12.0, most preferably from about 9.0 to about 11.0.

The acidic and basic pH conditions enable the coprecipitation of the filbrillar protein and the sulfated polysaccharide. The coprecipitation is further enhanced in the presence of a volatile, preferably non-toxic organic solvent. According to one embodiment, the volatile organic solvent is an alcohol.

According to one embodiment, the present invention provides a method for preparing the biocompatible cohesive biopolymer gel of the present invention comprising:

providing a solution of a fibrillar protein;

providing a solution of a sulfated polysaccharide;

combining the two solutions at appropriate pH in the absence of exogenous cross-linking agent to form a coprecipitate of cohesive biopolymer; and

precipitating the cohesive biopolymer with a volatile organic solvent.

According to preferred embodiments the coprecipitates may be formed or molded to any desired shape. According to certain embodiments the gels may be dried and stored prior to use. In these embodiments the gels are rehydrated with a suitable medium prior to use.

In order to provide the cohesive biopolymer coprecipitate with desired attributes, e.g. tensile strength, surface charge, density, porosity, ability to withstand suturing without tearing, etc., it is possible to add optional components either as a separate layer or interspersed or dispersed within the novel biopolymer of the invention.

It should be understood that the interaction between the sulfated polysaccharides and fibrillar protein resulting in the cohesive biopolymer coprecipitate of the invention may be covalent, non-covalent, or electrostatic. Cross-linked copolymers may provide further improvements to the product. According to optional features of the invention it may be advantageous to utilize cross-linking agents to alter or stabilize the attributes of the sulfated polysaccharide fibrillar protein coprecipitate. Cross-linking agents are known in the art and may include: simple sugars including pentoses or hexoses; aldehydes including glutaraldehyde; or synthetic cross-linkers if appropriate. According to one currently preferred embodiment, the cross linking agent is ribose.

According to a first aspect of the current invention we disclose an innovative material made of gelatin and dextran sulfate, having unique advantageous properties. The new cohesive biopolymer allows the preparation of articles of various shapes, including but not limited to tubes and sheets, and any other desired shape or form. When the shaped articles are not used immediately, they may be dehydrated for storage, and then re-hydrated prior to use.

As exemplified herein the novel coprecipitates disclosed according to the present invention are suitable for many clinical applications, including as a support or as a guide for peripheral nerve regeneration, as a sleeve for coating or enclosing the spinal cord, as a patch for repairing a lesion in a tissue, as a membrane for repairing tracheal lesions, as a coating or envelope for a vascular or tracheal stent.

The novel biopolymers of the invention are useful in the fabrication of medical devices, the form or shape of these devices depending on the specific intended use. The methods for fabrication of these devices may vary widely depending on the intended use.

These biopolymers are suited for use as fibers which fibers can be fabricated by conventional processes such as dry extrusion, gel extrusion, melt extrusion, solution extrusion or spinning extrusion, spraying of nanofibrils with or without an electromagnetic field, or by combination of these processes. The fibers can then be dried and spooled onto spools. The fibers can be woven, knitted, bundled or braided into complex form or constructs by methods known from industrial applications of textile manufacture.

The degree of solubility of the biopolymer matrices according to the present invention in various aqueous or organic solvents depends on the specific sulfated polysaccharide used to interact with the protein of choice, and on the coprecipitation conditions. According to some embodiments, the biopolymer of the invention disintegrates into metabolic degradable substances, which are soluble in aqueous solvents.

The biopolymer of the present invention is suited for extrusion and co-extrusion with different components, organic or inorganic in nature and polymeric or otherwise, including multiple components, multilayered types of fiber as well as hollow fibers and tubes.

According to one currently more preferred embodiment, dextran sulfate is coprecipitated with gelatin, to provide cohesive biopolymer gel with unexpectedly advantageous chemical and physical properties, in addition to its biological properties of biocompatibility, controllable biodegradation rate, affinity for cultured cells, and fostering cell growth. The novel cohesive biopolymer has physicochemical properties different from those of the uncombined raw materials, as can be evaluated by MRI analyses, infrared spectrum, elution from gel separation columns and other analytical tools known in the art.

According to a first embodiment of the invention, these biopolymers are useful per se as a biocompatible implant for guided tissue regeneration or tissue engineering. According to a further embodiment of the present invention these biopolymers are useful when they further comprise implants bearing cells to be transplanted to a site of injury or to ameliorate tissue impairment. According to a further embodiment of the present invention the cohesive biopolymer gels further comprise additional bioactive molecules to enhance tissue repair or regeneration.

Methods of using these cohesive biopolymers in vivo in clinical applications are disclosed, whereby the cohesive biopolymer gels according to the present invention may be used clinically either per se or as a scaffold for a cell bearing implant, alone or with additional layers of components. The cohesive biopolymers according to the present invention may advantageously be used as a substrate suitable for supporting cell selection, cell growth, cell propagation and differentiation in vitro as well as in vivo.

The cohesive biopolymers according to the invention comprise dextran sulfate in the range of about 30% to about 70% (w/w) and gelatin in the range of about 30% to 70% (w/w). This range of ingredients provides scaffold with the desired properties in terms of flexibility and elasticity. Typically, the biopolymer of the invention may conveniently be formed by interaction of approximately equal amounts of dextran sulfate and gelatin. According to one embodiment, the biopolymer of the invention is formed by interaction of 70% gelatin with 30% dextran sulfate.

The present invention also provides for the addition of ther active ingredients to the biopolymers comprising dextran sulfate and gelatin, including but not limited to other proteins such as fibrin, albumin, collagen, elastin and lysozyme; one of the diverse polysaccharides proteoglycans and hyaluronate; cross-linkers such as factor XIII, lysyloxidase; anticoagulants; growth factors; antioxidants and the like. These optional additives may be incorporated in such a manner to provide for desired pharmacokinetic profiles. Within the scope of the present invention there are provided methods of using the dextran sulfate-gelatin biopolymer gels for sustained release of bioactive components in vivo. In other instances the additives may be incorporated in such a manner to provide for short-lived optimal local concentrations of the bioactive molecules incorporated therein.

The physicochemical parameters of the cohesive biopolymer gel may readily be optimized in accordance with the intended use of the scaffold, and methods are disclosed to provide guidance to the skilled artisan in optimization.

The present invention is explained in greater detail in the description, figures and claims below.

### BRIEF DESCRIPTION OF THE FIGURES

FIG. 1, 2, 3 Different views of a nerve cuff made from a biopolymer of dextran sulfate combined with gelatin (GD biopolymer) according to the invention.

FIG. 4 Gel filtration profiles on Sepharose CL-6B column of a) dextran sulfate vs. coprecipitated biopolymer of gelatin and dextran sulfate (GD biopolymer) b) gelatin vs. GD biopolymer.

FIG. 5 Nuclear Magnetic Resonance spectra of a) dextran sulfate; b) gelatin; and c) coprecipitated biopolymer of gelatin and dextran sulfate.

FIG. 6 Infrared spectra of a) dextran sulfate; b) gelatin; and c) combined biopolymer of gelatin and dextran sulfate.

FIG. 7 Swelling degree of GD membranes NVR-3 in distilled water before and after cross-linking.

FIG. 8 Swelling degree of GD membranes NVR-3 in simulated saliva solution before and after cross-linking.

FIG. 9 Degradation rate of GD-membranes NVR-3 cross-linked with ribose over time.

FIG. 10 Porosity of dry GD biopolymer membrane, as shown by scanning electron microscopy.

FIG. 11 Embryonic rat spinal cord cells on NVR-7 tube after 45 days of growth. (magnification: ×100).

### DETAILED DESCRIPTION OF THE PREFERRED EMBODIMENTS

Sulfated Polysaccharide-Protein Coprecipitates

The present invention provides a biocompatible scaffold gel comprising a new polymeric material. The biopolymer of the present invention has unique chemical and mechanical properties as well as cell growth permissive features, and is therefore suitable for use as an implant, with or without cells.

Various gels or matrices generated from proteins in conjunction with other biocompatible polymers have shown great promise in the area of tissue engineering. According to the present invention we now disclose novel coprecipitates comprising at least one fibrillar protein and at least one sulfated polysaccharide. These novel coprecipitates are obtained in the absence of an exogenous cross linking agent, in the presence of a volatile organic solvent. The coprecipitates so obtained are subsequently shaped into any desired shape or geometry as required for a particular application. They may be further cross linked with an additional cross linking agent after the initial coprecipitate has formed. According to various embodiments various additives may be advantageously added to the gels prior to their formation or prior to use.

According to one exemplary embodiment the coprecipitates of the present invention comprise gelatin and dextran sulfate. Gelatin-Dextran sulfate (G-D) coprecipitates are useful for guiding tissue regeneration and as cell carriers in various clinical applications or other fields.

The polymers composing the cohesive biopolymer gel of the present invention were selected to simulate the two main constituents of the matrices of common connective tissues, namely collagens and glycosaminoglycans. The cohesive biopolymers gel of the present invention comprises a coprecipitate of sulfated polysaccharides and gelatin or other fibrillar proteins. According to one embodiment, sulfated polysaccharides include dextran sulfate, chondroitin sulfate, heparan sulfate, heparin, keratan sulfate, dermatan sulfate, as well as algal polyglycan sulfates, or synthetic sulfated polysaccharides as are known in the art. According to one currently preferred embodiment gelatin is used to mimic the collagen, and dextran sulfate is used to simulate the glycosaminoglycans in forming the biopolymer gel of the present invention. The term “gel” is used herein in the conventional sense to refer to water-swellable polymeric matrices that can absorb a substantial amount of water to form elastic gels. The gel may be hydrated to obtain a dry gel. Upon placement in an aqueous environment, dry gels swell to the extent allowed by the degree of the interactions between the gel-forming polymers.

Dextran is a glucose polymer and dextran sulfate is a polysaccharide, composed of sulfated glucose as the repeating units. It contains about 17%-20% sulfur with up to three sulfated groups per glucose molecule. The molecular weight of dextran sulfate is in the range of 4,000 to 500,000 KD. As used herein, the term “molecular weight” refers to the weight of average molecular weight of a number of molecules in any given sample, as commonly used in the art. Thus, a sample of Dextran sulfate 5,000 kD might contain a statistical mixture of polymer molecules ranging in weight from, for example, 4,500 to 5,500 kD, with one molecule differing slightly from the next over a range. Variations in the dextran sulfate molecular weight are associated with differences in its biological activity.

Use of sulfated polysaccharides of various molecular weights and applying different coprecipitation conditions in producing the gelatin-sulfated polysaccharides biopolymer of the present invention result in a gel having different properties.

Use of a high molecular weight dextran sulfate in the production of GD matrix according to the present invention results in a readily biodegradable matrix. Use of low molecular weight dextran sulfate prolongs the retention time of the biopolymer gel in vitro and in vivo, and results in a biopolymer with increased strength and elasticity.

According to one embodiment of the present invention the dextran sulfate has a molecular weight of from about 4,000 Dalton to about 500,000 Dalton. According to another embodiment, the GD biopolymer of the present invention is produced with a high molecular weight dextran sulfate having a molecular weight of more than 300,000 Dalton, preferably more than 400,000 Dalton, most preferably with dextran sulfate having a molecular weight of about 500,000 Dalton.

According to alternative embodiment, the GD bioploymer of the present invention is produced with dextran sulfate of low molecular weight having a molecular weight of below 10,000 Dalton, preferably below 8,000, most preferably dextran sulfate having a molecular weight of about 5,000 Dalton.

According to the present invention dextran sulfate of any source may be used, including dextran sulfate commercially available, synthetically prepared, or isolated from natural source. According to one embodiment, the dextran sulfate used according to the present invention is of bacterial origin, which best simulates the glycosaminoglycans.

Gelatin is a heterogeneous mixture of water-soluble proteins of high average molecular weight. Gelatin is not found in nature but is derived form collagen by hydrolytic action. Gelatin is obtained by boiling skin, tendons, ligaments, bones etc., with water. Approximate amino acid content of gelatin is: glycine—25.5%, alanine 8.7%, valine 2.5%, leucine 3.2%, isoleucine 1.4%, cystine and cysteine 0.1%, methionine 1.0%, phenylalanine 2.2%, threonine 1.9%, tyrosine 0.5% aspartic acid 6.6% glutamic acid 11.4%, arginine 8.1% lysine 4.1% and histidine 0.8%. The total is over 100% because water is incorporated into the molecules of individual acids.

Though it is possible, according to the present invention, to use gelatin obtained from human collagen, more preferred are materials of non-human origin, due to safety concerns related to the use of collagen obtained from human sources. According to one embodiment, the gelatin is of mammalian species other than human. According to one currently preferred embodiment the gelatin to be used is porcine or bovine gelatin.

The coprecipitation of dextran sulfate and gelatin is generally performed in a controlled manner, i.e. using a specific ratio of dextran sulfate to gelatin, dextran sulfate of certain molecular weight, specific pH conditions and certain incubation time, thus controlling the degree of interaction between to two polymers. The coprecipitate is formed in the absence of an exogenous cross-linking agent in the presence of a volatile organic solvent. The resulted GD cohesive biopolymer of the invention comprises dextan sulfate in the range of from about 30% to about 70% (w/w) and gelatin in the range of about from 30% to about 70% (w/w). This range of ingredients provides scaffold with the desired properties in terms of flexibility, elasticity and biodegradability. According to one embodiment, the biopolymer of the invention is formed by coprecipitating approximately equal amounts of dextran sulfate and gelatin. According to another embodiment, the biopolymer of the invention is formed by interaction of 70% gelatin with 30% dextran sulfate. The interaction between the dextran sulfate and gelatin resulting in the coprecipitated biopolymer of the present invention rnay be covalent, non-covalent or electrostatic.

The properties of the cohesive biopolymer gel of the present invention also depend on the conditions under which the coprecipitation of dextran sulfate and gelatin is performed, particularly the pH range of the interaction medium.

According to one embodiment, the coprecipitate is formed at a pH of at least 2 pH units above or below natural pH. According to one embodiment, the coprecipitation of gelatin and dextran sulfate of high molecular weight is performed under acidic pH conditions. According to one currently preferred embodiment, the coprecipitation of gelatin and dextran sulfate having molecular weight of above 300,000 Dalton is performed at a pH range of from about 2.0 to about 5.0, preferably from about 2.0 to about 4.0.

According to alternative embodiment, the coprecipitation of gelatin and dextran sulfate of low molecular weight is performed under basic pH conditions. According to one embodiment, the coprecipitation of gelatin and dextran sulfate having a molecular weight below 10,000 Dalton is performed at a pH range of from about 8.0 to about 12.0, preferably from about 9.0 to about 12.0, most preferably from about 9.0 to about 11.0.

The novel cohesive biopolymer gel of the present invention is obtained by the coprecipitation of gelatin and dextran sulfate from the aqueous solution under extreme pH conditions, either acidic or basic. To further enhance this spontaneous coprecipitation, a volatile organic solvent may be added to the solution. According to one embodiment, the volatile organic solvent is an alcohol, particularly ethanol. The resulted coprecipitate is removed from the solution and it may be then first shaped or directly dehydrated. The matrix may be dried at ambient air temperature or at elevated temperature. During the dehydration process the organic solvent evaporates; thus, no chemical reagents that might impair the bioavailability of the resulting gel are present in the final product. According to one embodiment, the volatile organic solvent is non-toxic, and preferably is an alcohol. The dehydrated matrix is ready for storage, and should be re-hydrated prior to use.

According to one embodiment, the present invention provides a method for preparing the biocompatible cohesive biopolymer gel of the present invention comprising:

providing a solution of a fibrillar protein;

providing a solution of sulfated polysaccharide;

combining the two solutions at appropriate pH in the absence of exogenous cross-linking agent to form a coprecipitate of cohesive biopolymer; and

precipitating the cohesive biopolymer with a volatile organic solvent.

In order to provide the coprecipitated matrix with other desired attributes, e.g. tensile strength, surface charge, density, porosity, ability to withstand suturing without tearing, etc., it is possible to add optional components either as a separate layer or interspersed or dispersed within the novel biopolymer of the invention.

According to one embodiment, the gelatin-dextran sulfate matrix of the present invention can also be further cross-linked to alter or stabilize the attributes of the GD polymer by means of any of a number of conventional chemical cross-linking agents, including, but not limited to simple sugars including pentoses or hexoses, glutaraldehyde, divinyl sulfone, epoxides, carbodiimides, and imidazole. The concentration of chemical cross-linking agent required is dependent on the specific agent being used and the degree of cross-linking desired. The cross linked polymer may offer increased reproducibility as well as other improvements to the product, including increased retention time in vitro and in vivo and increased strength. According to one currently preferred embodiment, the cross-linking agent is ribose.

The novel cohesive biopolymer of the present invention has physicochemical properties different from those of the uncombined raw material. As exemplified herein below, characterization of the GD biopolymer by gel filtration chromatography, nuclear magnetic resonance spectroscopy (NMR) and infrared spectroscopy shows that the GD coprecipitate cohesive biopolymer according to the present invention is clearly distinguished from gelatin or dextran sulfate alone.

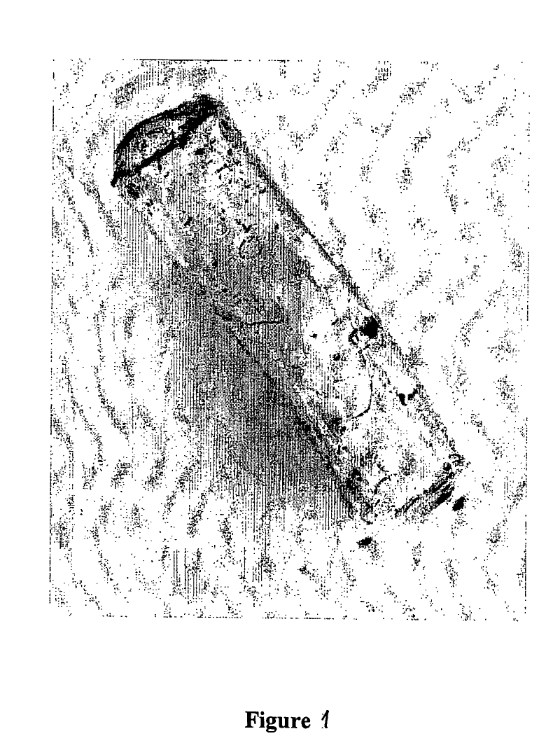

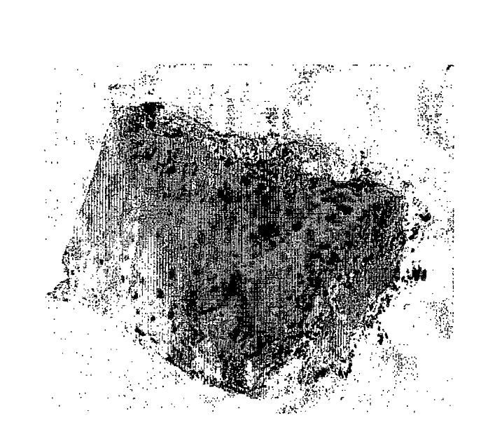

The GD cohesive biopolymer is easily sterilized and stored at room temperature, capable of large-scale production and moldable into various shapes, including but not limited to tubes and sheets suitable for the support of a guide for peripheral nerve regeneration, a sleeve for coating or enclosing the spinal cord, a patch for repairing a hole in a tissue, particularly closing tracheal holes, and a coating or envelope for a vascular and tracheal stent. The biopolymer is useful in the fabrication of medical devices, the form or shape of these devices depending on their intended use. The method for fabrication of these devices may vary widely depending on the intended use. The ability to control the physicochemical properties of the GD biopolymer according to the present invention allow for the broad range of shapes and utilities of this scaffold biopolymer gel. The GD-biopolymer could be designed to be more rigid or more flexible; readily biodegradable or with elongated retention time; insoluble in water or with moderate solubility, and the like. As exemplified herein below, the GD biopolymer was shaped into a tube, that designed to help restore function to patients with peripheral nerve injures (whole sectional loss) by acting as a bridge for guiding the nerve regeneration (also see FIGS. 1, 2, 3). A shape of a membrane was also designed, and the membrane was used to cover a hole in a rabbit trachea. The membrane was sewed onto the trachea, and the specific GD biopolymer type used enabled the sewing without rupturing the membrane.

The biopolymer is suited for use as fibers which fibers can be fabricated by conventional processes such as dry extrusion, gel extrusion, melt extrusion, solution extrusion or spinning extrusion or by combination of these processes. The fibers can then be dried and spooled onto spools. The fibers can be woven, knitted, bundled or braided into complex form or constructs by methods known from industrial applications of textile manufacture.

The biopolymer is suited for extrusion and co-extrusion with different components, organic or inorganic in nature and polymeric or otherwise, including multiple components, multilayered types of fiber as well as hollow fibers and tubes.

By using suitable methods as are known in the art it is possible to optimize this material for biocompatibility, cytotoxicity aspects, and other desired parameters including rate of biodegradability, tensile strength of fibers, flexibility of sheets and tubes, porosity, etc.

For controlling the biodegradation rate of the cohesive biopolymer products (i.e., to increase or decrease their biodegradability) it is possible to add a polymerizable macromolecule with known biocompatibility and known degradation time, exemplified but not limited to collagen, polyurethane, polyglycolic/polylactic acids, trimethylene carbonate, among others. The improved material with for example incorporated carbonate and/or dioxanone linkages are selected to improve various properties of the material, particularly increasing viscosity, viscoelasticity and retention time, while prolonging yet preserving biodegradability.

Furthermore, we provide methods for allowing the presence of pores within the biopolymer material and for determining the preferred pore sizes. Pores may be desirable in relation to stimulation of cell adhesion, growth, and differentiation, and in the converse intactness may be needed for certain applications such as for formation of a tracheal stent.

According to one embodiment, the matrix of the present invention fabricated in either of the shapes described above, is useful per se as a biocompatible implant for use in a variety of medical applications, including, but not limited to vascular grafts, artificial organs, heart valves and for guided tissue regeneration or tissue engineering.

According to another embodiment these matrices are useful when they further comprise implant bearing cells to be transplanted to a site of injury or to ameliorate tissue impairment. The cohesive biopolymers according to the present invention may advantageously be used as a substrate suitable for supporting cell selection, cell growth, cell propagation and differentiation in vitro as well as in vivo.

According to a further embodiment of the present invention the matrices further comprise additional bioactive molecules to enhance tissue repair or regeneration. As used herein, bioactive molecules describe molecules exemplified by, but not limited to growth factors, cytokines and active peptides (which may be either naturally occurring or synthetic), which aid in the healing or re-growth of normal tissue. The function of such bioactive molecules include stimulating local cells to produce new tissue and/or attracting cells to the cite in need of correction. Biologically active molecules useful in conjunction with the cohesive polymer of the present invention include, but are not limited to, cytokines: interferons (IFN), tumor necrosis factors (TNF), interleukins, colony stimulating factors (CSFs); growth factors: osteogenic factor extract (OFE), epidermal growth factor (EGF), transforming growth factor (TGF) alpha, TGF-β (including any combination of TGF-β), TGF-β1, TGF-β2, platelet derived growth factor (PDGF-AA, PDGF-AB, PDGF-BB), acidic fibroblast growth factor (FGF), basic FGF, connective tissue activating peptides (CTAP), β-thromboglobulin, insulin-like growth factors, erythropoietin (EPO), and nerve growth factor (NGF); proteins: fibrin, albumin, collagen, elastin and lysozyme; The term “bioactive molecules” as used herein is further intended to encompass drugs such as antibiotics, anti-inflammatories, antithrombotics, and the like; one of the diverse polysaccharides proteoglycans and hyaluronate; cross-linkers such as factor XIII, lysyloxidase; anticoagulants; and antioxidants. These optional additives may be incorporated in such a manner to provide for desired pharmacokinetic profiles. Within the scope of the present invention there are provided methods of using the dextran sulfate-gelatin biopolymer gels for sustained release of bioactive components in vivo. In other instances the additives may be incorporated in such a manner to provide for short-lived optimal local concentrations of the bioactive molecules incorporated therein.

The following examples are intended to illustrate the principles of the invention and are to be construed in a non-limitative manner.

### EXAMPLES

### Example 1

### Manufacture of the GD Biopolymer

A. Gelatin-High Molecular Weight Dextran Sulfate Biopolymer, Acidic pH—NVR-3

A solution of Gelatin (20 mg/ml in Hanks Balanced Salt Solution (HBSS) from Gibco, Catalog #14025-50) and a solution of dextran sulfate (M.W. 500,000 Dalton, 10 mg/ml in HBSS) were mixed at 70° C. in the proportion of 70% of gelatin to 30% of dextran sulfate by weight (w/w), so that the final concentrations are 20 mg/ml of gelatin and 10 mg/ml of dextran sulfate. The ratio of 70/30 (w/w) of gelatin to dextran sulfate was found optimal for this combination of gelatin-dextran sulfate. After 3 min., the pH of the solution was adjusted to 3.0 using 5N acetic acid (0.1 ml of acetic acid solution per 1 ml of mixture), and the solution was mixed carefully by shaking. After additional 3 min. a coprecipitate gel was formed and it was further precipitated from the solution with absolute ethanol. The unpolymerized molecules remain soluble, while the cohesive biopolymer is removed from liquid as a precipitate. The precipitated gel was collected and removed from the solution by a spatula. The collected gel-like matrix was air-dried at room temperature, until the matrix reached a constant weight. The dry gel-like material was immersed in a 1% ribose in 80% ethanol solution and incubated at room temperature for 7 days for further cross-linking. Alternatively, the biopolymer can be cross-linked by thermal treatment or by other chemical agents, for example, acetone, ethyl-3(3-dimethyl amino) propyl carbodiimide (EDC) oxidizing agents that are capable of forming active groups like aldehydes. For example sodium periodate is capable of forming aldehydes readily reactive with free amino group followed by reduction with sodium borohydride. The final polymer has a high viscosity, almost semisolid, with a high wet tensile strength around 70-75 MPa and high resistance to mechanical cutting (e.g., by a surgical suture of 20N).

B. Gelatin-Low Molecular Weight Dextran Sulfate Biopolymer, Acidic pH—NVR-5

A solution of gelatin (20 mg/ml in HBSS) and a solution of dextran sulfate (M.W. 5,000 Daltons, 20 mg/ml in HBSS) were mixed at 70° C. in the proportion of 50/50 by weight, so that the final concentrations were 5 mg/ml of gelatin and 5 mg/ml of dextran sulfate. The mixture was incubated for 3 min. The pH was then adjusted to pH 3.0 using 5N acetic acid (0.1 ml of acetic acid solution per 1 ml of the polymeric mixture). The dispersion was mixed carefully by shaking for additional 3 min. The formed coprecipitate gel was further precipitated with absolute ethanol and removed by a spatula. The polymeric gel was dried under ambient temperature until a constant weight for reached. The dry biopolymer gel was further incubated in 1% ribose solution in 80% absolute ethanol for 7 days for the formation of additional cross-linking.

The traces of acidity remained after the process of preparation of NVR-3 and NVR-5 interferes with the compatibility of the gel as a cells-bearing matrix. The resulted gel was relatively soluble in aqueous solution, and therefore readily degradable. NVR-6 and NVR-7 were therefore produced, with low molecular weight dextran sulfate.

C. Gelatin-High Molecular Weight Dextran Sulfate Biopolymer, Basic pH—NVR-6

A solution of Gelatin (20 mg/ml HBSS) and a solution of dextran sulfate (M.W. 500,000 Dalton, 20 mg/ml in HBSS) were mixed in the proportion of 50% of gelatin to 50% of dextran sulfate by weight (w/w), so that the final concentrations are 10 mg/ml of gelatin and 10 mg/ml of dextran sulfate. The pH was adjusted to 11.0 using 10N ammonium hydroxide (0.0125 ml ammonium hydroxide per 1 ml of the mixture). The mixture was agitated at 120-150 rpm for 24 h at room temperature. The formed coprecipitate gel was further precipitated with absolute ethanol, and than removed from the solution by a spatula The resulted gel was dried in ambient temperature, and was found to be insoluble in aqueous solutions.

D. Gelatin-Low Molecular Weight Dextran Sulfate Biopolymer—NVR-7

A solution of gelatin (20 mg/ml in HBSS) and a solution of dextran sulfate (M.W. 5,000 Daltons, 20 mg/ml in HBSS) were mixed in the proportion of 50/50 by weight, so that the final concentrations were 10 mg/ml of gelatin and 10 mg/ml of dextran sulfate. The pH was adjusted to 11.0 using 10N ammonium hydroxide (0.0125 ml ammonium hydroxide per 1 ml of the mixture). Alternatively, another bases may be used, for example diisopropylenamine. The mixture was agitated at 120-150 rpm for 24 h at room temperature. The formed coprecipitate gel was further precipitated with absolute ethanol, and than removed from the solution by a spatula. Typically, the resulted NVR-7 matrix showed high strength, and therefore an additional cross-linking was not applied. However, if a stronger matrix is desired, cross-linking can be performed as described above.

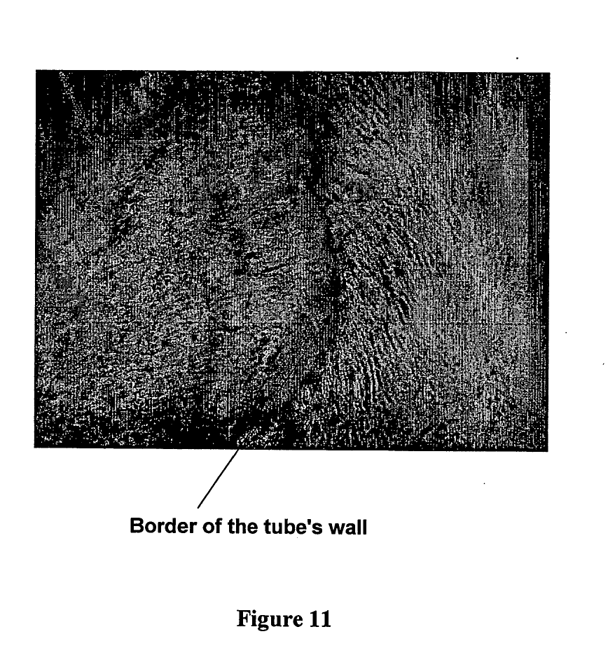

All the NVR products described above were found to be biocompatible both in vitro and in vivo, and are therefore useful in the fabrication of medical devices, the form or shape of these devices depending on the intended use, and the method for fabrication also depending on the use may vary widely. The NVR-3 and NVR-5 biopolymers were found to be less suitable for cell growth, specifically neuronal cell growth, probably due to a high density of negative SO₄⁻² charge, while NVR-6 and NVR-7 were found to be highly suitable for cell growth. For example, embryonic rat spinal cord cells were found to grow successfully and sprout on the surface of the construct (FIG. 11). The NVR-7 matrix, shaped as a membrane was found to be intact after 45 days of cell growth. No interference to the cell growth by the membrane was observed.

The biopolymers are also suited for use as fibers, which can be fabricated by conventional processes such as dry extrusion, gel extrusion, melt extrusion, solution extrusion or spinning extrusion or by combination of these processes. The fibers can then be dried and spooled onto spools. The fibers can be woven, knitted, bundled or braided into complex form or constructs by methods known from industrial applications of textile manufacture.

The biopolymer is suited for extrusion and co-extrusion with different components, organic or inorganic in nature and polymeric or otherwise, including multiple components, multilayered types of fiber as well as hollow fibers and tubes.

### Example 2

### Forming the GD-Tube

Cohesive biopolymer gels obtained as described in Example 1 above were transferred to a glass Petri dish and heated at 100° C. for 1-2 hrs in a dry oven until ethanol was completely evaporated. Then the polymeric substance was cooled to room temperature and transferred to a hot (100° C.) mold for preparation of sleeves or membranes by a compression molding procedure. After cooling the formed item was removed from the mold and dried to a constant weight.

When NVR-7 was produced, it was found to be water insoluble and thus has a longer retention time to biodegradation compared to NVR-3 and NVR-5. Incubation of NVR-7 matrix in sterile PBS at 37° C. for 4 month did not cause any changes in the construct appearance, integrity and/or weight.

### Example 3

### Testing the GD Tube to Serve as a Stent-Sleeve, or Coating

A GD-Tube in the length of 5 mm with a diameter of 2 mm was stretched over a balloon carrying a coronary stent. The balloon was inflated to 16 atmospheres with water. The sleeve remained intact under two inflation cycles of 16 atmospheres. This ability of the cohesive biopolymer for stretching displays its potential for serving as stent-sleeve to lower restenosis and thrombosis rates after angioplasty.

### Example 4

### Use of the GD Tube for Enclosing Neuronal Implant

The methodologies for the maintenance, growth and differentiation of neuronal cultures are known to be most sophisticated. Therefore, an extracellular matrix (ECM) milieu that mimics the in vivo substrate and requirements of neuronal cells is most desirable.

Tissue culture methods have gained attention as a substitute for the use of in vivo animal models. One direction was devoted to the creation and simulation in vitro of the in vivo environment, nature and composition of the extracellular matrix (ECM) for the cultured cells or explants. As disclosed previously by one of the present inventors (WO 02/39948), two major components, namely Hyaluronic acid (HA) and Laminin (LN), have emerged as essential candidates specially for neuronal and glial cell cultures.

The combination of HA and LN into one viscous adhesive gel (HA-LN gel) has provided a biomatrix for growing neuronal cells and explants that derived from both the central and the peripheral nervous systems. The combination of HA and LN, which are major components of the ECM have been introduced by the inventors as substrates for growing neuronal cells and explants derived from both the central and peripheral nervous systems.

It was disclosed previously (WO 02/39948) that in addition to providing a useful substrate matrix for a broad range of cell types in vitro, the HA-LN gel serves as a highly advantageous biocompatible implant and as delivery vehicle for transplantation.

Nevertheless, it turns out that in order to improve the mechanical properties it is desirable to enclose the HA-LN gels in a more rigid scaffold prior to implantation into a patient. This is achieved by use of the dextran sulfate-gelatin cohesive biopolymer of the present invention as a scaffold enclosing the HA-LN gel implant, the latter with our without cells.

The NVR guiding tube (denoted herein as GD tube) will be filled with the NVR-N-Gel with or without cells.

### Example 5

### Use of the GD Membrane for Closing a Tracheal Hole

Air leakage, structure collapse, flow obstructions, airways occlusions, vascular compression, hypotonicity, myoelasticity and respiratory distress are characteristic symptoms in pediatrics laryngo-tracheo-bronchomalacia and/or stenosis.

Bronchomalacia is due to cartilaginous deficiency in the tracheal or bronchial wall, occurring in children under six months. There is collapse of a mainstem bronchus, on expiration. There are two types of Bronchomalacia Primary bronchomalacia is due to a deficiency in the cartilaginous rings. Secondary bronchomalacia may occur by extrinsic compression from an enlarged vessel, a vascular ring or a bronchogenic cyst. Both types may results in compressive lesions that may be identified by MRI or CT examination. Bronchomalacia is a life-threatening illness in pediatric medicine.

Similar symptoms of Bronchomalacia may results from prolonged intubation (tube therapy), which can induce intra-tracheal scarring and fibrosis, leading to the above fatal pathologies.

NVR-7 membrane was examined as a treatment of deformed airways. The experiment was performed in vitro, in lung taken form a healthy rabbit, as well as in vivo, by deliberately forming a cut in the rabbit lung trachea. After the cut was formed, an NVR-7 membrane of 1.0 cm×0.5 cm was sewed to cover the cut. In both experiments the membrane clogged completely the airway system, and enable a normal function of the lung. After the surgery, the rabbit was able to breath normally.

### Example 5

### Characterization of the GD Biopolymer

The GD biopolymer is produced as a coprecipitate of two simple polymeric molecules: dextran sulfate and gelatin. A series of tests were performed to compare the original raw materials and the new formed cohesive biopolymer gel, as described herein below:

Gel Filtration Chromatography (GFC)

Gel filtration chromatography (GFC) profile of substances depends on the molecular weight as well as the 3-D shape of the molecule. Ten mg of polymeric substance dissolved in double distilled water was placed on the column of Sepharose CL-6B and eluted with double distilled water. The excluded (void) volume was determined employing dextran blue of 2×10⁶ Daltons, eluted in tube # 7 (11.5 ml) 1.5 ml/tube.

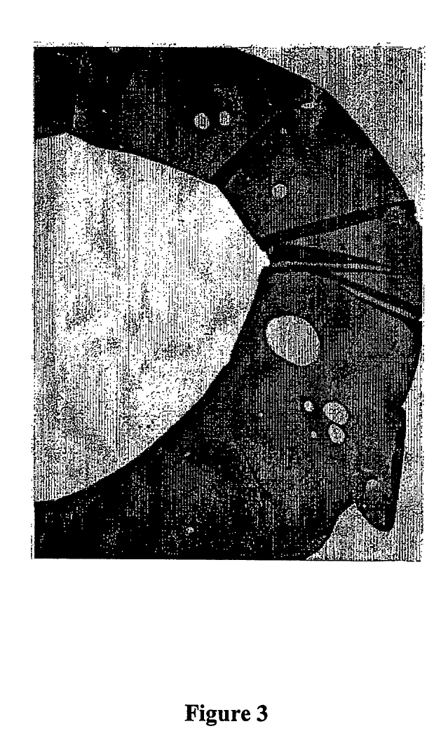

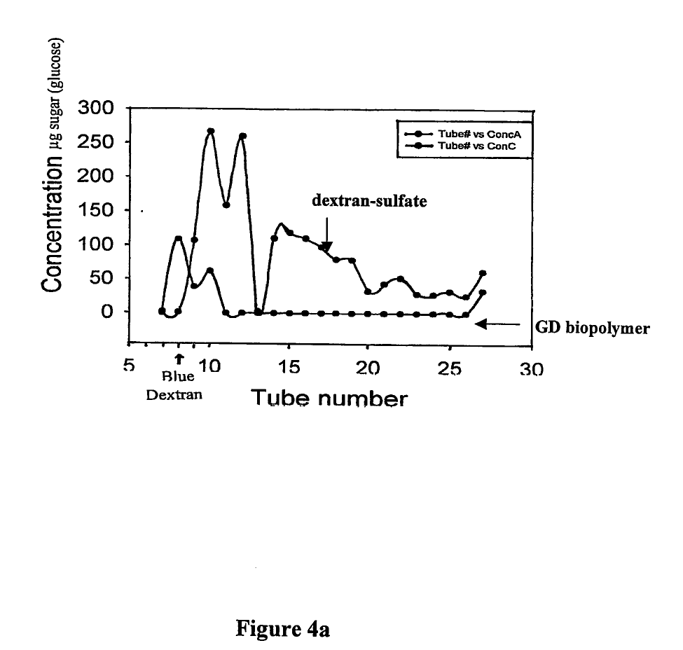

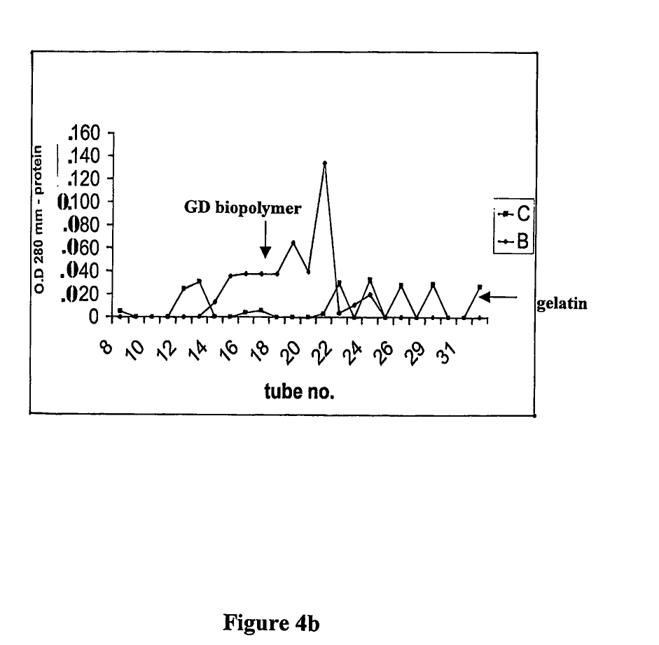

Polysaccharides of 1×10⁶ and up were excluded. Sugars were followed by the phenol method for neutral sugars (1 ml sample+1 ml 5% phenol solution+5 ml of concentrated sulfuric acid). The orange color developed was read in a spectrophotometer at a wavelength of 490 μm. As shown in FIG. 4, the chromatogram of dextran sulfate alone (FIG. 4a) and the chromatogram of the novel GD biopolymer (FIG. 4c) are notably different.

Similarly, gel filtration chromatography was performed to compare gelatin alone and the GD biopolymer.

Ten mg of polymeric substance dissolved in 0.5 ml of DMSO was placed on Sepharose CL-6B column. Elution was performed with a 10% DMSO solution, collecting fractions of 1.5 ml each. Proteins having molecular weights of 4×10⁶ Daltons and above were eluted at the void volume. Proteins were detected by spectrophotometer at a wavelength of 280 nm. Again, the chromatogram profile of gelatin (FIG. 4b) is distinctly different from the profile of the biopolymer (FIG. 4c).

Nuclear Magnetic Resonance Spectroscopy (NMR)

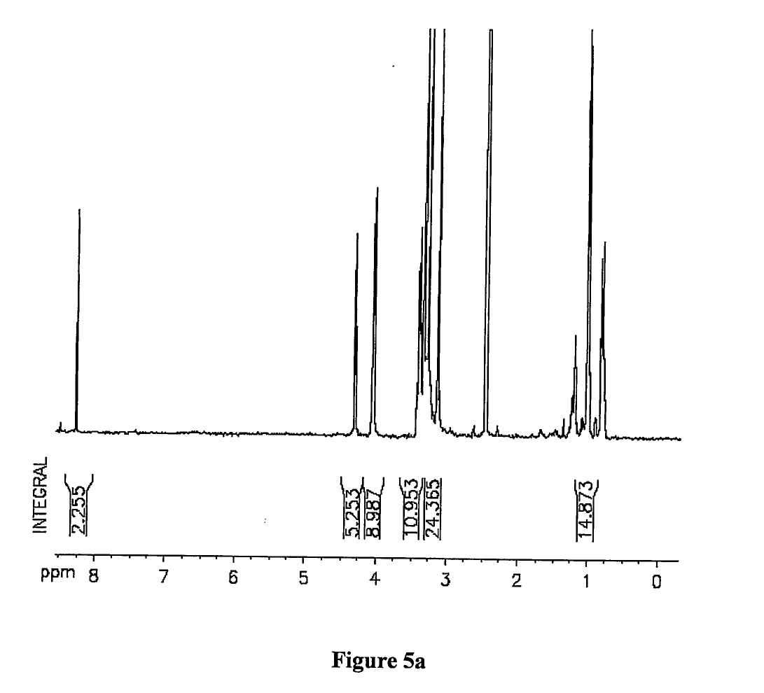

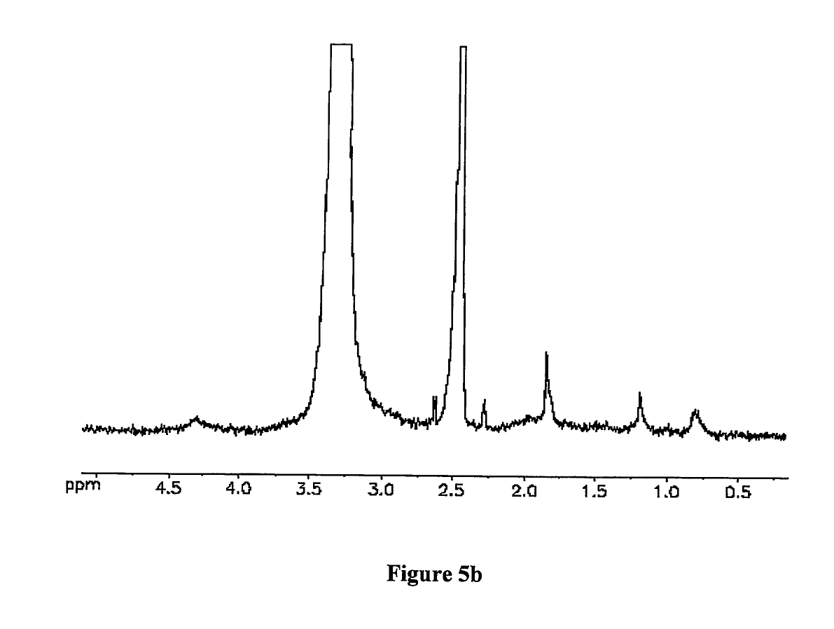

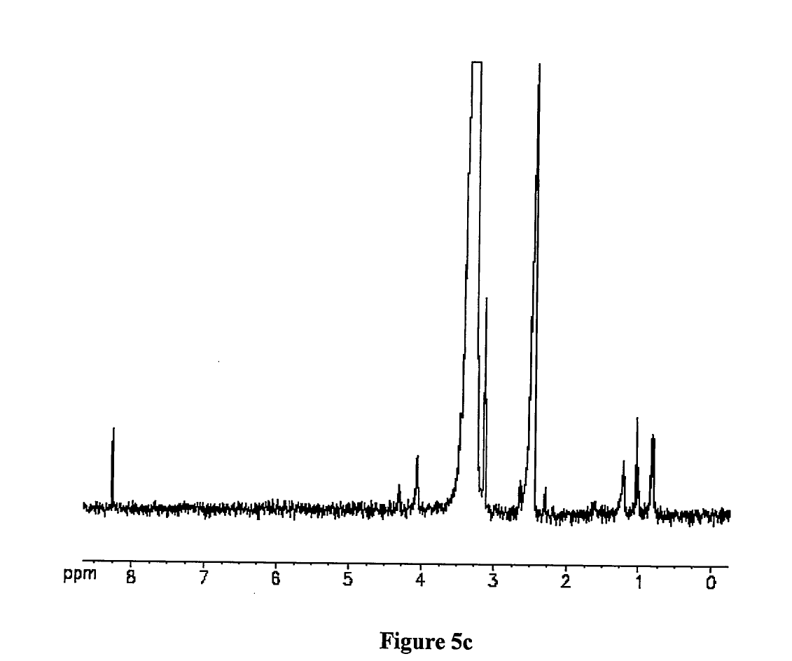

FIG. 5 a-c describe NMR analyses of gelatin, dextran sulfate and the novel GD biopolymer. The results clearly show that the new biopolymer is distinguished from the original raw materials.

Infra Red Spectrometry

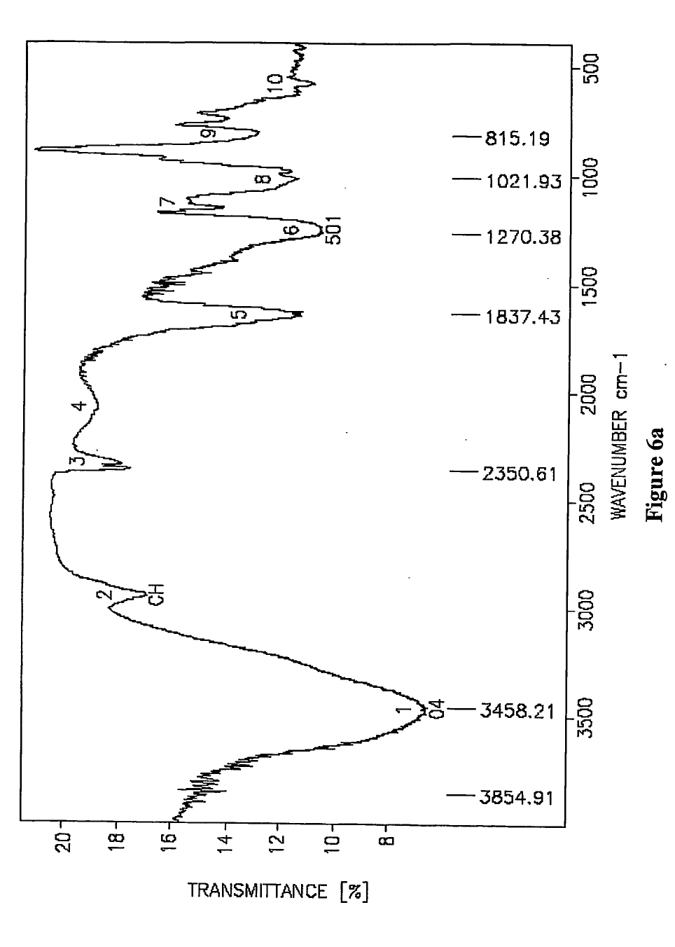

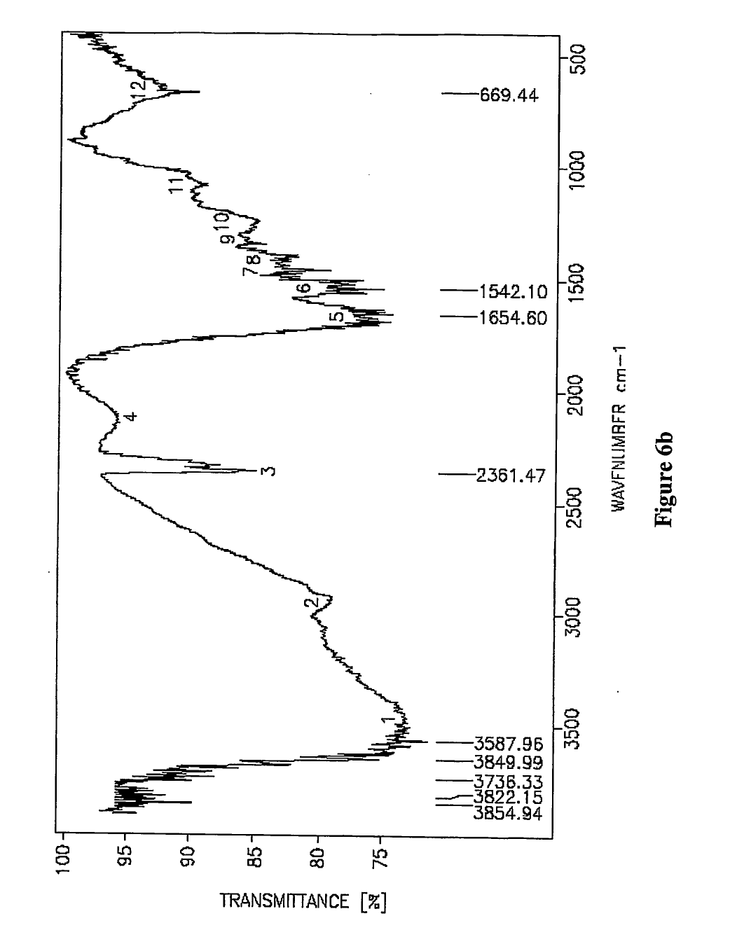

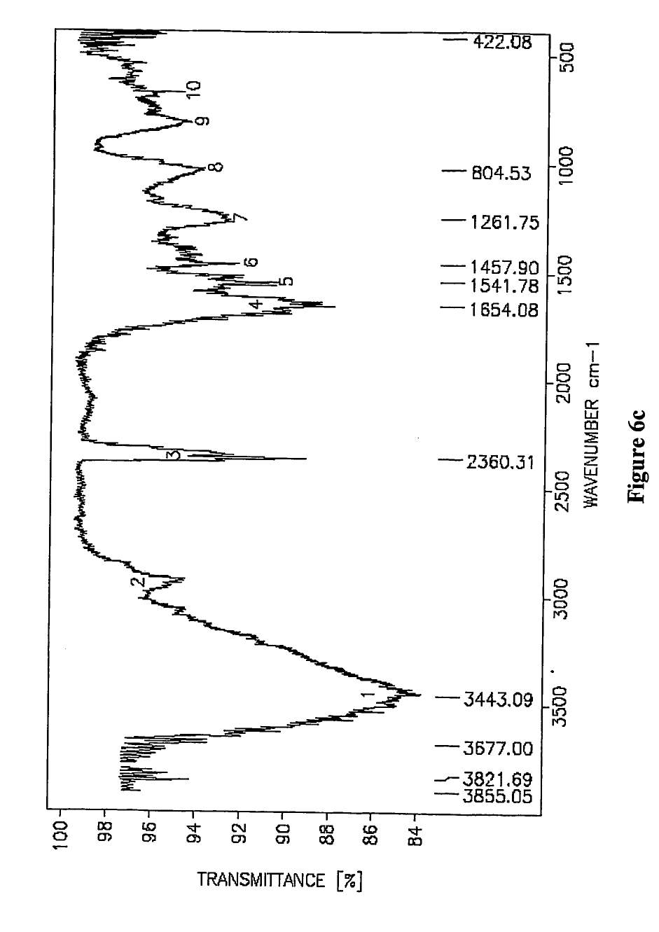

FIG. 6 shows the infrared spectra of gelatin and dextran sulfate as raw materials in comparison with the spectrum of the GD biopolymer.

A gross analysis of the spectrum, showing that the spectrum of the GD biopolymer differ from those of the raw material molecules, suggests the formation of a new polymeric substance, as was shown by the results of the other tests described above.

### Example 6

### Characterization of GD Biopolymers with Different Degrees of Cross-Linking

The degradation rate of the polymer can be controlled by the extent and type of the cross-linking between the polymer molecules. The method of the present invention, reacting the biopolymer with reducing sugars, resulted in intensive cross-linking of the biopolymer molecules. Comparing a full hand of cross-linking agents showed pentose monosaccharide ribose as the best agent. The degree of cross-linking is controllable by the sugar concentration, temperature and the length of the reaction.

Comparing the GD properties before and after cross-linking examined the influence of the cross-linking degree on the properties of the GD biopolymer.

Swelling Test

The membranes swelling studies were conducted using two media, namely, distilled water and simulated saliva solution. Each sample of membrane (NVR-3, surface area 40 mm²) was dried by vacuum for 4 h, weighed and placed in a pre-weighed stainless steel wire mesh with sieve openings of approximately 200 μm. The mesh sieve with the film sample was submerged into 25 ml medium placed in a plastic beaker. Increase in weight of the membranes was determined at successive time intervals until a constant weight was obtained. Each measurement was repeated three times. The degree of swelling was calculated using the following parameters:

$$\frac{{W}_{t} - {W}_{0}}{{W}_{0}} \times 100 \%$$

Where Wₜ is the weight of the membrane at time t; and W₀ is the weight of membrane at time zero.

The samples were tested before cross-linking, after cross-linking by dehydrothermal treatment and after cross-lining by a combination of dehydrothermal treatment and sugars.

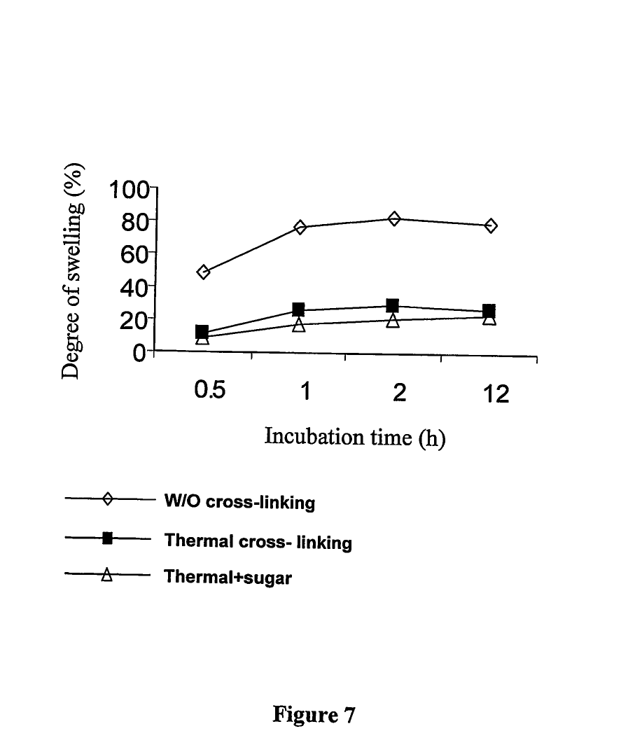

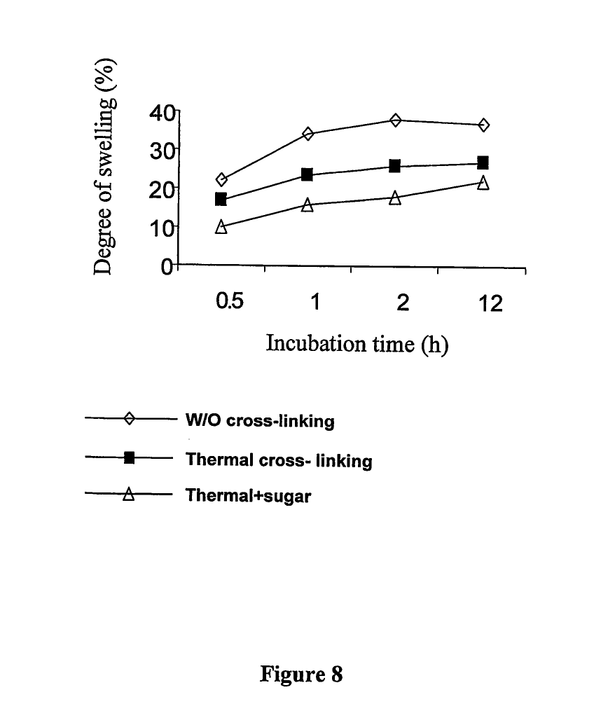

FIGS. 7 and 8 depict the degree of swelling of the GD membranes in distilled water and simulated saliva solution, respectively, before and after cross-linking. The degree of swelling was higher in distilled water compared to saliva solution. This finding suggests that ionic strength and pH play an important role in affecting the swelling of the membranes. The rate of swelling for the GD membranes before cross-linking was higher compare to membranes after thermal cross-linking and significantly lower for membranes after the combination of thermal treatment with cross-linking by sugars. These data indicate that both the thermal method and the sugars cross-linking decrease the rate of water uptake and hydration, as a result of increasing the degree of cross-linking.

In Vitro Biodegradation

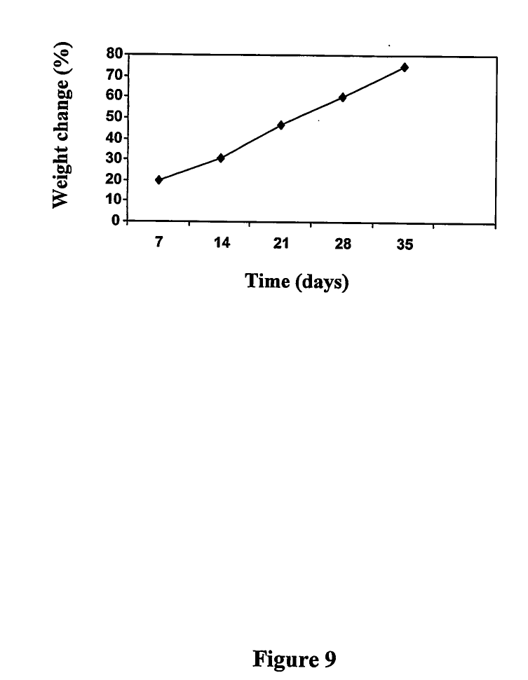

Gelatin-dextran sulfate-membranes (GD membranes, NVR-3) were prepared and cross-linked by ribose. Samples, prepared for testing, were dried by vacuum for 4 h, weighed and fully immersed in the physiological solution, supplemented with 20% fetal bovine serum at 37° C. for the specified period of time. At each specified time period throughout the duration of the incubation time, the solution was replaced and samples were removed, dried and weighed. By measuring the weight change during the 30 days duration of the assay, the degradation rate was calculated. FIG. 9 shows that the ribose cross-linked preparations are degraded at a rate of 2-2.5% per day.

### Example 7

### Porosity of the GD Biopolymer

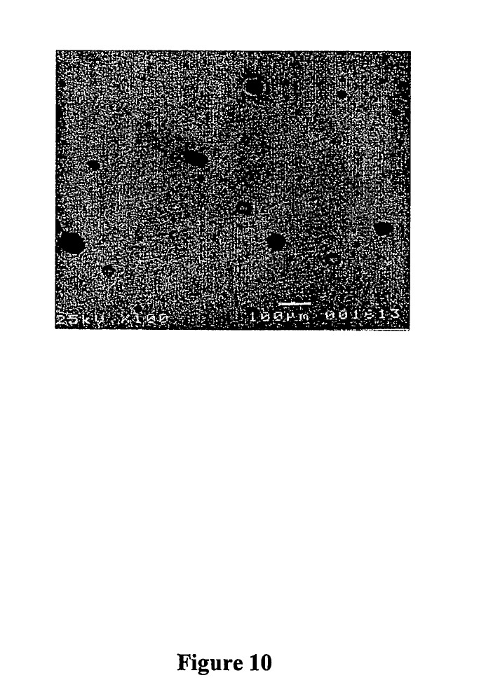

The structure of a dry GD membrane (NVR-3) was examined by Scanning Electron Microscope (SEM), before and after incubation of the membrane in neuronal cell culture medium for 24 days. As shown in FIG. 10, the dry membrane appears as a continuous dense solid with a randomly porous structural; the size of the pores was 20-70 μm.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

### REFERENCES

Christensen N. D., Reed C. A. Culp T. D., Hermonat P. L. Howett M. K., Anderson R. A., and Zanevell L. J. Papillamavirus microbicidal activities of high-molecular-weight cellulose sulphate, dextran sulphate, and polystyrene sulfonate. Antimicrobial Agents Chemother. 45, 3427-3432, 2001.
Chupa J. M., Foster, A. M., Sumner, S. R., Madhally, S. V., Matthews, H. W. Vascular cell responses to polysaccharide materials: in vitro and in vivo evaluation. Biomaterials 21, 2315-2322, 2000.
Cohlbery J. A., Li, J. Uversky V. N. and Fink A. L. Heparin and other glycosaminoglycans stimulate the formation of amyloid fibrils from alpha-synuclein in vitro. Biochemistry 41, 1502-1511, 2002.
de Chalain, T., Phillips J., Hinek, A. Bioengineering of elastic cartilage with aggregated porcine and human auricular chondrocytes and hydrogels containing alginate, collagen, and kappa-elastin. J. Biomed. Mater. Res. 44, 280-288, 1999.
Fujiyama J., Sakakura C., Takagi T., Itoh T., Takemura M., Touma A., Shimmoura K., Yunning H., Longhai H., Tsujimoto H., Yamasaki J., Otsuji E., Hagiwara A., and Yamagishi H. Analysis of the action of dextran sulphate as a prophylaxis for peritoneal cancer metastasis, and its acting mechanism, Gan To Kagaku. Ryoho 28, 1677-1680, 2000.
Laurent T C. The interaction between polysaccharides and other macromolecules. The exclusion of molecules from hyaluronic acid gels and solutions. Biochem. J. 93, 106-112 1964.
Ibanoglu E. High pressure effect on foaming properties of beta-lactoglobulin and dextran sulphate mixture. Nahrung 45, 342-346, 2001.
Risbud M., Ringe J., Bhonde R., and Sittunger M. In vitro expression of cartilage specific markers by chondrocytes on a biocompatible hydrogel: Implications for engineering cartilage tissue. Cell Transp. 10, 755-763, 2001.
Robinson M. J., Tessier P., Poulsom R., and Hogg N. The S-100 family heterodimer, MRP-8114, links with high affinity to heparin and haparan sulphate glycosaminoglycans on endothelial cells. J. Biol. Chem. 277, 3658-3665, 2002.
Wang, P. Y. and Bazos, M. J. Polymer surface interaction in the biological environment. J. Controlled Release, 2, 47-57, 1985.

1-42. (canceled)

43. A biocompatible cohesive biopolymer gel comprising a coprecipitate of at least one fibrillar protein and at least one sulfated polysaccharide.

44. The biocompatible cohesive biopolymer gel of claim 43 wherein the coprecipitate is formed in the absence of an exogenous cross-linking agent in the presence of a volatile organic solvent.

45. The biocompatible cohesive biopolymer gel of claim 43 wherein the coprecipitate is formed at a pH of at least 2 pH units above or below neutral pH.

46. The biocompatible cohesive biopolymer gel of claim 45 wherein the coprecipitate is formed at an acidic pH, between pH of about 2.0 and pH of about 5.0.

47. The biocompatible cohesive biopolymer gel of claim 45 wherein the coprecipitate is formed at a basic pH between pH of about 9.0 and pH of about 12.0.

48. The biocompatible cohesive biopolymer gel of claim 43 wherein the protein is selected from the group consisting of collagen, elastin, fibrin, albumin and gelatin.

49. The biocompatible cohesive biopolymer gel of claim 48 wherein the protein is gelatin.

50. The biocompatible cohesive biopolymer gel of claim 43 wherein the sulfated polysaccharide is selected from the group consisting of dextran sulfate, chondroitin sulfate, heparin, heparan sulfate, keratan sulfate, dermatan sulfate, algal sulfated polyglycan, or a synthetic sulfated polysaccharide.

51. The biocompatible cohesive biopolymer gel of claim 50 wherein the sulfated polysaccharide is dextran sulfate.

52. The biocompatible cohesive biopolymer gel of claim 51 wherein the dextran sulfate has a molecular weight in a range of from about 4,000 Dalton to about 500,000 Daltons.

53. The biocompatible cohesive biopolymer gel of claim 52 wherein the dextran sulfate is selected from
(a) a high molecular weight dextran sulfate having a molecular weight in a range of from about 300,000 Dalton to about 500,000 Dalton; and
(b) a low molecular polymer dextran sulfate having a molecular weight in a range of from about 5,000 Dalton to about 10,000 Dalton.

54. The biocompatible cohesive biopolymer gel of claim 43 wherein the cohesive biopolymer comprises gelatin and dextran sulfate.

55. The biocompatible cohesive biopolymer gel of claim 54 comprising 30% to 70% of dextran sulfate.

56. The biocompatible cohesive biopolymer gel of claim 54 comprising 30% to 70% of gelatin.

57. The biocompatible cohesive biopolymer gel of claim 54 comprising about 50% gelatin and about 50% dextran sulfate.

58. The biocompatible cohesive biopolymer gel of claim 43 further comprising at least one substance selected from anticoagulants, adhesive molecules, growth factors, enzymes, antioxidants, antifibrotic substances, positively charged molecules, a peptide rich in positively charged amino acids, and nutritional elements.

59. The biocompatible cohesive biopolymer gel of claim 44 further comprising bridges formed by subsequent addition of a cross-linking agent to the coprecipitate formed.

60. The biocompatible cohesive biopolymer gel of claim 59 wherein the cross-linking agent is selected from the group consisting of a monosaccharide: ribose, glucose, mannose and xylose; factor XIII; lysyloxidase; a carbodiimide; and an oxidizing agent.

61. The biocompatible cohesive biopolymer gel of claim 43 further comprising at least one bioactive compound selected from the group consisting of a hormone, a growth factor, a proteolytic enzyme, an anti-fibrotic agent, a coagulative agent, an extracellular matrix component, an anti oxidant, a natural or synthetic polymer.

62. The biocompatible cohesive biopolymer gel of claim 43 wherein the coprecipitate is formed into fibers, sheets, sponges, fabrics or tubes.

63. The biocompatible cohesive biopolymer gel of claim 43 wherein the coprecipitate is formed as a coating for a stent, wherein the stent is selected from a vascular stent and a tracheal stent.

64. The biocompatible cohesive biopolymer gel of claim 43 wherein the coprecipitate is formed into a scaffold.

65. The biocompatible cohesive biopolymer gel of claim 64 wherein the scaffold encloses hyaluronic acid-laminin gel.

66. The biocompatible cohesive biopolymer gel of claim 43 wherein the coprecipitate is formed into a scaffold for enclosing neuronal cells.

67. The biocompatible cohesive biopolymer gel of claim 66 wherein the scaffold enclosing neuronal cells further encloses hyaluronic acid-laminin gel.

68. The biocompatible cohesive biopolymer gel of claim 43 formed into a scaffold for use as a cell bearing implant.

69. An implant comprising a biocompatible cohesive biopolymer gel according to claim 43.

70. A method for preparing a biocompatible cohesive biopolymer gel suitable as an implant in a human or animal, which comprises:
providing a solution of a fibrillar protein;
providing a solution of sulfated polysaccharide;
combining the two solutions at a pH of at least 2 pH units above or below neutral pH in the absence of an exogenous cross-linking agent to form a coprecipitate of cohesive gel; and
precipitating the cohesive gel with a volatile organic solvent.

71. The method of claim 70 wherein the fibrillar protein is gelatin.

72. The method of claim 70 wherein the sulfated polysaccharide is dextran sulfate.

73. The method of claim 72 wherein the dextran sulfate has a molecular weight in a range of from about 4,000 Dalton to about 500,000 Daltons.

74. The method of claim 73 wherein the dextran sulfate is selected from
(a) a high molecular weight dextran sulfate having a molecular weight in a range of from about 300,000 Dalton to about 500,000 Dalton; and
(b) a low molecular polymer dextran sulfate having a molecular weight in a range of from about 5,000 Dalton to about 10,000 Dalton.

75. The method of claim 70 wherein the pH is an acidic pH between pH of about 2.0 and pH of about 5.0.

76. The method of claim 70 wherein the pH is a basic pH between pH of about 9.0 and pH of about 12.0.

77. The method of claim 70 wherein the volatile organic solvent is an alcohol.

78. The method of claim 70 further comprising forming the gel into fibers by a process selected from the group consisting of dry extrusion, gel extrusion, melt extrusion, solution extrusion, spinning extrusion, spraying of nanofibrils and combinations thereof.

79. The method of claim 70 further comprising shaping the biocompatible cohesive biopolymer gel.

80. The method of claim 70 further comprising incorporating a bioactive substance into the biopolymer.

81. A kit for carrying out extemporaneously a method according to claim 70, the kit comprising at least one dose of each constituent solution necessary to obtain the coprecipitate which forms the biocompatible cohesive biopolymer gel.

82. A composition for sustained release of a bioactive substance comprising a bioactive substance within a biocompatible cohesive biopolymer gel according to claim 43.

83. A method for repairing a lesion in a tissue, comprising applying the biocompatible cohesive biopolymer gel of claim 43 at a site of the lesion.

84. The method of claim 83 wherein the lesion is a tracheal lesion.
